(19) United States
(12) Patent Application Publication
(10) Pub. No.: US 2024/0261242 A1
(43) Pub. Date: Aug. 8, 2024

(54) METHODS OF INTRAVENOUSLY ADMINISTERING SOTALOL

(71) Applicant: AltaThera Pharmaceuticals LLC, Chicago, IL (US)

(72) Inventor: Brandon Ira Kashfian, Chicago, IL (US)

(21) Appl. No.: 18/631,538

(22) Filed: Apr. 10, 2024

Related U.S. Application Data
(63) Continuation-in-part of application No. 18/324,703, filed on May 26, 2023.
Application No. 18/324,703 is a continuation-in-part of application No. 17/585,190, filed on Jan. 26, 2022, now abandoned.
Application No. 17/585,190 is a continuation of application No. 16/863,567, filed on Apr. 30, 2020, now abandoned.
Application No. 16/863,567 is a continuation-in-part of application No. 16/693,312, filed on Nov. 24, 2019.
Application No. 16/693,312 is a continuation of application No. 16/103,815, filed on Aug. 14, 2018, now Pat. No. 10,512,620.
Application No. 16/863,567 is a continuation-in-part of application No. 16/693,310, filed on Nov. 24, 2019, now Pat. No. 11,696,902.
Application No. 16/693,310 is a continuation-in-part of application No. 16/103,815, filed on Aug. 14, 2018, now Pat. No. 10,512,620.
Application No. 18/324,703 is a continuation-in-part of application No. 17/306,490, filed on May 3, 2021.
Application No. 17/306,490 is a continuation of application No. 16/849,099, filed on Apr. 15, 2020, now abandoned.
Application No. 17/306,490 is a continuation-in-part of application No. 16/693,312, filed on Nov. 24, 2019.
Application No. 16/693,312 is a continuation-in-part of application No. 16/693,310, filed on Nov. 24, 2019, now Pat. No. 11,696,902.
(60) Provisional application No. 63/346,003, filed on May 26, 2022.
(60) Provisional application No. 63/009,511, filed on Apr. 14, 2020.
(60) Provisional application No. 62/987,832, filed on Mar. 10, 2020.

(51) Int. Cl. A61K 31/18; A61K 9/00; A61P 9/06

(52) U.S. Cl. CPC A61K 31/18; A61K 9/0019; A61P 9/06

(57) ABSTRACT

Methods of administering one or more IV doses of sotalol hydrochloride to a patient in need thereof are described. In embodiments, one or more of the IV doses of sotalol hydrochloride are administered as substitute(s) for one or more oral dose(s) of sotalol hydrochloride, with the substitute IV dosing based on the subject's creatinine clearance and when repeated as a selected interval is capable of providing a sotalol concentration commensurate with the steady state concentration expected from multiple sequential oral doses.

### CROSS REFERENCE TO RELATED APPLICATIONS

The present application is a Continuation-in-Part application of U.S. patent application Ser. No. 18/324,703, filed May 26, 2023, which application claims priority to and the benefit of the filing date of U.S. Provisional Patent Application No. 63/346,003, filed May 26, 2022. The '703 application is a Continuation-in-Part application of U.S. patent application Ser. No. 17/585,190, filed Jan. 26, 2022, which application is a Continuation application of U.S. patent application Ser. No. 16/863,567, filed Apr. 30, 2020. The '567 application claims priority to and the benefit of the filing date of U.S. Provisional Patent Application No. 63/009,511, filed Apr. 14, 2020, and is a Continuation-in-Part application of U.S. patent application Ser. No. 16/693,312, filed Nov. 24, 2019. The '312 application is a Continuation application of U.S. patent application Ser. No. 16/103,815, filed Aug. 14, 2018, which issued as U.S. Pat. No. 10,512,620 on Dec. 24, 2019. The '567 application is a Continuation-in-Part application of U.S. patent application Ser. No. 16/693,310, filed Nov. 24, 2019, which issued as U.S. Pat. No. 11,696,902 on Jul. 11, 2023. The '310 application is a Continuation-in-Part application of U.S. patent application Ser. No. 16/103,815, filed Aug. 14, 2018, which issued as U.S. Pat. No. 10,512,620 on Dec. 24, 2019. The '703 application is a Continuation-in-Part application of U.S. patent application Ser. No. 17/306,490, filed May 3, 2021, which application is a Continuation application of U.S. patent application Ser. No. 16/849,099, filed Apr. 15, 2020, which application claims priority to and the benefit of the filing date of U.S. Provisional Patent Application No. 62/987,832, filed Mar. 10, 2020. The '490 application is a Continuation-in-Part application of U.S. patent application Ser. No. 16/693,312, filed Nov. 24, 2019. The '312 application is a Continuation application of U.S. patent application Ser. No. 16/103,815, filed Aug. 14, 2018, which issued as U.S. Pat. No. 10,512,620 on Dec. 24, 2019. The '490 application is a Continuation-in-Part application of U.S. patent application Ser. No. 16/693,310, filed Nov. 24, 2019, which issued as U.S. Pat. No. 11,696,902 on Jul. 11, 2023. The '310 application is a Continuation-in-Part application of U.S. patent application Ser. No. 16/103,815, filed Aug. 14, 2018, which issued as U.S. Pat. No. 10,512,620 on Dec. 24, 2019. The disclosures of these applications are hereby incorporated by reference herein in their entireties.

### BACKGROUND OF THE INVENTION

### Field of the Invention

The present invention relates to the field of cardiovascular pharmaceutics, more particularly compositions and methods for the treatment of cardiovascular conditions, such as arrhythmias, with intravenous anti-arrhythmics, such as sotalol hydrochloride.

### Description of Related Art

Sotalol hydrochloride is a racemic mixture of d- and l-sotalol hydrochloride. Sotalol hydrochloride is a white, crystalline solid with a molecular weight of 308.8. It is hydrophilic, soluble in water, propylene glycol, and ethanol. Chemically, sotalol hydrochloride is a d,1-N-[4-[1-hydroxy-2-[(1-methylethyl)amino]ethyl]phenyl] methane-sulfonamide monohydrochloride. (HIGHLIGHTS OF PRESCRIBING INFORMATION: Sotalol hydrochloride injection for intravenous use, Revised June 2009). The molecular formula is C12H2ON203S•HCl and is represented by the following structural formula:

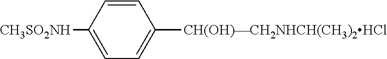

Intravenous sotalol can be supplied as a sterile, clear solution in 10 ml vials, each vial containing 150 mg sotalol hydrochloride in acetate buffer. Sotalol hydrochloride is an antiarrhythmic drug with both beta adrenoreceptor blocking (Class II) and cardiac action potential duration prolongation (Class III) properties. The drug is hemodynamically well-tolerated and has a small risk of proarrhythmia. Both isomers have similar Class III activity, while the 1-isomer is responsible for virtually all of the beta blocking activity. Sotalol does not have partial beta agonist or membrane stabilizing activity (Na channel inhibition). Sotalol does not undergo metabolism and is nearly 100% bioavailable when taken on an empty stomach. Sotalol hydrochloride does not bind to plasma proteins. The pharmacokinetics of d- and l-sotalol enantiomers are essentially identical. Excretion is predominantly via the kidney in the unchanged form and therefore lower doses are necessary in patients with renal impairment.

Sotalol is an effective antiarrhythmic agent but also can cause serious ventricular arrhythmias, primarily Torsade de Pointes (TdP) type ventricular tachycardia, a polymorphic ventricular tachycardia associated with QTc prolongation. QTc prolongation is directly related to the plasma concentration of sotalol. Conventionally, steady-state plasma levels of sotalol and maximum QTc prolongation are obtained in 3 days with oral administration (i.e. after 5-6 doses when administered twice daily). QTc changes are directly related to maximum serum concentration of the sotalol. To minimize the risk of sotalol caused arrhythmias, the product label mandates that patients initiated or re-initiated on sotalol should be hospitalized for at least three days or until steady state drug levels are achieved, in a facility that can provide cardiac resuscitation and continuous electrocardiographic monitoring.

Hospitalization represents a high-cost burden in the United States. The three-day hospital stay is also burdensome to the patient, resulting in loss of productivity, time away from family, and an increased risk of hospital-acquired infections, as well as a diversion of hospital resources, staff, and patient care beds. The availability of IV sotalol offers an opportunity to improve on IV sotalol protocols and/or decrease the length of hospital stay.

Sotalol is FDA approved for the maintenance of normal sinus rhythm in patients with history of highly symptomatic atrial fibrillation/flutter and for the treatment of documented life-threatening ventricular arrhythmias. Sotalol is currently approved in the US for oral administration (for example, under the brand name BETAPACE AF®, Bayer HealthCare Pharmaceuticals Inc.) and is approved for IV administration (AltaThera Pharmaceuticals LLC).

Sotalol may be implemented in the treatment and/or management of a variety of cardiovascular conditions, including for example, one or more of atrial flutter (AFL), atrial fibrillation, persistent atrial fibrillation, paroxysmal atrial fibrillation, ventricular fibrillation (VF), ventricular arrhythmias, ventricular tachycardia (VT), premature ventricular contractions (PVCs), paroxysmal atrial tachycardia, focal atrial tachycardia, supraventricular tachycardia (SVT), accessory pathway SVT, infantile SVT, post-operative SVT, paroxysmal supraventricular tachycardia (paroxysmal SVT), atrioventricular re-entrant tachycardia (AVRT), atrioventricular nodal re-entrant tachycardia (AVNRT), atrial tachycardia, junctional tachycardia, junctional ectopic tachycardia (JET), multifocal atrial tachycardia, atrial ectopic tachycardia, hemodynamically stable or unstable ventricular tachycardia, congenital heart disease (CHD), heart failure, coronary artery disease, and pulmonary artery hypertension. (See, e.g., patent literature relating to IV sotalol, including U.S. Pat. Nos. 10,512,620, 10,799,138, 11,344,518, 11,583,216 and 11,610,660; and US Published Patent Application Nos. 2019/0307343, 2019/0380605, 2020/0085771, 2020/0093759, 2020/0253903, 2020/0338027, 2021/0076959, 2021/0283049, 2022/0142954, 2022/0241225, 2022/0339130, 2023/0075398, 2023/0172883, 2023/0187049, 2023/0225997, 2023/0248674, 2023/0255908, 2023/0270940, 2023/0293455 and 2023/0372268, which are each individually incorporated by reference herein in their entireties).

Sotalol hydrochloride is typically administered orally; however, methods of IV administration are needed for a variety of reasons (e.g., NPO patients, patients with gastrointestinal conditions resulting in poor absorption, patients recovering from GI surgery, and/or patients in intensive care). A need remains for methods of administering IV sotalol hydrochloride as a substitute for an oral sotalol hydrochloride dosing regimen, which can be used to address these needs or can be used as desired in place of an oral sotalol dosing regimen.

### SUMMARY OF THE INVENTION

Described herein are methods of administering sotalol hydrochloride to a subject in need thereof in an amount effective for treating a cardiovascular condition. Sotalol is a commonly used oral anti-arrhythmic drug for management of atrial arrhythmias. This disclosure provides a change from the oral administration guidelines for sotalol. Thus, the present methods can enable physicians to treat patients with conditions incompatible with traditional oral sotalol dosing.

Aspects of the invention include Aspect 1, which is a method of intravenously administering sotalol, comprising: determining a creatinine clearance of a subject; selecting an oral sotalol hydrochloride dosing regimen capable of providing a desired effect in the subject as a result of multiple sequential oral doses; selecting an IV dose comprising an amount of sotalol hydrochloride that, when based on the subject's creatinine clearance and repeated at a selected interval, is capable of providing a sotalol concentration commensurate with the steady state concentration expected from the multiple sequential oral doses; and for treatment of a cardiovascular condition and as a substitute for the oral dosing regimen, administering to the subject the IV dose repeated at the selected interval to achieve the sotalol concentration commensurate with the steady state concentration expected from the multiple sequential oral doses.

Aspect 2 is a method of substituting oral sotalol with intravenous sotalol hydrochloride, comprising: a. inputting a subject's creatinine clearance into a treatment management application, such as inputting one or more parameters (gender, age, serum creatinine, weight, height) for the treatment management application to calculate the subject's creatinine clearance; b. inputting into the treatment management application an oral sotalol hydrochloride dosing regimen capable of providing a desired effect when administered to the subject as multiple sequential oral doses; c. the treatment management application providing an output comprising an IV dose comprising an amount of sotalol hydrochloride that when based on the subject's creatinine clearance and repeated at a selected interval is capable of providing a sotalol concentration commensurate with the steady state concentration expected from the multiple sequential oral doses; and d. for treatment of a cardiovascular condition and as a substitute for the oral dosing regimen, administering to the subject the IV dose repeated at the selected interval to achieve the sotalol concentration commensurate with the steady state concentration expected from the multiple sequential oral doses. In embodiments, creatinine clearance is based on gender, age and weight.

Aspect 3 is the method of Aspect 1 or 2, wherein: the amount of each sotalol hydrochloride IV dose for a first subject with mild renal impairment and a creatinine clearance in the range of 60-89 mL/min is the same as for a second subject with normal renal function and a creatinine clearance in the range of ≥90 mL/min; and the selected dosing interval is the same for the first and second subjects; whereby no adjustment as to IV dosing amount or interval is needed to accommodate the subject with mild renal impairment relative to the subject with normal renal function in order to achieve the sotalol concentration commensurate with the steady state concentration associated with and expected from the multiple sequential oral doses; optionally wherein the oral sotalol hydrochloride dosing regimen comprises oral doses in the amount of 80 mg, or 120 mg, or 160 mg.

Aspect 4 is the method of any of Aspects 1-3, wherein the IV dosing protocol is selected from any one or more of the protocols outlined below in Tables 1 and/or 2.

Aspect 5 is the method of any of Aspects 1-4, wherein the subject/patient is currently in sinus rhythm, or is not currently in sinus rhythm, or has been or will be converted to sinus rhythm with sotalol or another antiarrhythmic and/or cardioversion, such as electric cardioversion (e.g., DCCV or direct current cardioversion), and sotalol hydrochloride is administered for the treatment of one or more cardiovascular condition selected from atrial flutter (AFL), atrial fibrillation (AF), persistent atrial fibrillation, paroxysmal atrial fibrillation, paroxysmal or persistent atrial fibrillation or atrial flutter, such as for patients who have experienced a recent AF/AFL episode who are in sinus rhythm or who will be cardioverted, ventricular fibrillation (VF) or ventricular arrhythmias, life-threatening recurrent VF or life-threatening recurrent hemodynamically unstable VT, focal atrial tachycardia, supraventricular tachycardia (SVT), atrioventricular re-entrant atrioventricular nodal re-entrant tachycardia (AVRT), tachycardia (AVNRT), atrial tachycardia, paroxysmal atrial tachycardia, junctional tachycardia, junctional ectopic tachycardia (JET), congenital heart disease (CHD), multifocal atrial tachycardia, atrial ectopic tachycardia, accessory pathway SVT, infantile SVT, post-operative SVT, paroxysmal SVT, treating Wolff-Parkinson-White syndrome, ventricular tachycardia (VT), premature ventricular contractions (PVCs), hemodynamically stable or unstable ventricular tachycardia, heart failure, coronary artery disease, and pulmonary artery hypertension, maintenance of normal sinus rhythm (for example, delay in time to recurrence of atrial fibrillation/atrial flutter (AF/AFL)) for example in patients with symptomatic AF/AFL and/or who are currently in sinus rhythm, and/or indicated for the treatment of life-threatening ventricular tachycardia; and/or treating people, such as hospitalized people, with atrial fibrillation cardioverted to normal sinus rhythm, without ventricular arrhythmias or various forms of blocks, in patients with normal kidney function. In embodiments, sotalol can be administered to a patient who is receiving extracorporeal membrane oxygenation (ECMO).

Aspect 6 is the method of any of Aspects 1-5, wherein the subject is currently in sinus rhythm and/or has experienced cardioversion to sinus rhythm, for example by way of electrocardioversion, such as DCCV, or chemical cardioversion, such as with sotalol hydrochloride, or combinations thereof.

Aspect 7 is the method of any of aspects 1-6, wherein: the oral sotalol hydrochloride dosing regimen comprises 80 mg oral doses; the creatinine clearance of the subject is in the range of 10-59 mL/min; the IV dose is administered in an amount of 67.5 mg.

Aspect 8 is the method of any of Aspect 1-6, wherein: the oral sotalol hydrochloride dosing regimen comprises 120 mg oral doses; the creatinine clearance of the subject is in the range of 10-59 mL/min; the IV dose is administered in an amount of 105 mg.

Aspect 9 is the method of any of Aspects 1-6, wherein: the oral sotalol hydrochloride dosing regimen comprises 160 mg oral doses; the creatinine clearance of the subject is in the range of 30-59 mL/min; the IV dosage is administered in an amount of 142.5 mg.

Aspect 10 is the method of any of Aspects 1-6, wherein: the oral sotalol hydrochloride dosing regimen comprises 160 mg oral doses; the creatinine clearance of the subject is in the range of 10-29 mL/min; the IV dose is administered in an amount of 135 mg.

Aspect 11 is the method of any of Aspects 1-10, wherein the selected interval is a 12-hour, 24-hour or 48-hour interval.

Aspect 12 is the method of any of Aspects 1-11, wherein the IV dose is a 5-hour intravenous dose.

Aspect 13 is the method of any of Aspects 1-12, wherein optionally one or more or multiple IV doses is replaced by an oral dose (e.g., an oral dose is administered followed by an IV dose, optionally repeated any number of times; or an IV dose is administered followed by an oral dose, optionally repeated any number of times; or an IV dose is followed by multiple oral doses, optionally repeated any number of times; or an oral dose is followed by multiple IV doses, optionally repeated any number of times; or an oral dose is followed by one or more or multiple IV doses, optionally repeated any number of times; or an IV dose is followed by one or more or multiple oral doses, optionally repeated any number of times; or only IV doses are administered; or multiple IV doses are administered followed by an oral dose, optionally repeated any number of times; or multiple oral doses are administered followed by an IV dose, optionally repeated any number of times; or multiple IV doses are administered followed by an oral dose followed by one or more IV dose followed by an oral dose, etc.; or multiple oral doses are administered followed by an IV dose followed by one or more oral dose followed by an IV dose, etc.).

Aspect 14 is the method of any of Aspects 1-13, wherein the sotalol concentration of the patient is commensurate with a steady state concentration expected from multiple sequential oral doses when the sotalol concentration is in the range of ±5% or ±10% or ±15% or ±20% of the expected steady state concentration.

### DETAILED DESCRIPTION OF VARIOUS EMBODIMENTS OF THE INVENTION

Reference will now be made in detail to various illustrative implementations. It is to be understood that the following discussion of implementations is not intended to be limiting.
AF is atrial fibrillation.
AFL is atrial flutter.
AF/AFL is atrial fibrillation and/or atrial flutter.
IV is intravenous.
PO means “per os” and refers to an oral dosing regimen.
BID means “bis in die” and means twice a day.
QD means “quaque die” and means once a day.
QID means “quater in die” and means four times a day.
Patient (or subject) refers to a human patient.
BP is blood pressure.
HR is heart rate.
CLCR or CrCL refers to creatinine clearance.

Renally impaired refers to patients having creatinine clearance rates of below 90 mL/min, including below 60 mL/min, such as below 30 mL/min. A subject with mild renal impairment can have a creatinine clearance in the range of 60-89 mL/min, and a subject with moderate renal impairment can have a creatinine clearance in the range of 30-59 mL/min, and a subject with severe renal impairment can have a creatinine clearance in the range of 10-29 mL/min.

Initiation of a subject on sotalol typically refers to the subject who is naïve to sotalol or who has not received sotalol for at least five (5) half-lives of sotalol.

Escalation means increasing the sotalol dosage of a patient already receiving sotalol, for example where a subject is currently taking a specific amount of an oral dose and escalation involves administering one or more IV doses to escalate the sotalol concentration of the subject to a higher level, typically followed by one or more subsequent IV dose. In embodiments, the IV escalation dose is initiated at a time when the next oral dose would have been due, such as when the sotalol concentration of the patient would be in a trough.

Sotalol and sotalol hydrochloride (used interchangeably herein) refer to d,l-sotalol hydrochloride.

The terms “treat,” “treating,” and “treatment” refer to performing a method, such as administration of a drug, for the purpose of treating or ameliorating an injury, disease or condition, including methods resulting or not resulting in any indicia of success in the treatment or amelioration of an injury, disease, or condition, including any objective or subjective parameter such as abatement; remission; diminishing of symptoms or making the injury disease, or condition more tolerable to the subject; slowing in the rate of degeneration or decline; or improving a subject's well-being, such as physical or mental. The treatment or amelioration of symptoms can be based on objective or subjective parameters, including the results of a physical examination and/or other patient evaluation.

Cₘₐₓss is the maximal concentration obtained at steady state.

QT is the interval measured from the start of the Q wave or QRS complex, to the end of the T wave, where the Q wave corresponds to the beginning of ventricular depolarization and the T wave end corresponds to the end of ventricular repolarization.

QTc is the calculated interval that represents the QT interval corrected for heart rate and can be derived by mathematical correlation of the QT interval and the heart rate.

ΔQTc is the difference between a QTc measurement taken prior to the start of treatment and a QTc measured after the start of treatment (e.g., during or after one or more IV dose).

The term “about” used herein in the context of quantitative measurements means the indicated amount ±10%. For example, “about 2 mg” can mean 1.8-2.2 mg.

Hospital refers to a medical facility staffed and equipped to provide continuous ECG monitoring and cardiac resuscitation to patients, if needed. Typically, the medical personnel are trained in the management of serious ventricular arrhythmias.

Sotalol is indicated for the maintenance of normal sinus rhythm (delay in time to recurrence of atrial fibrillation/atrial flutter (AFIB/AFL)) in patients with symptomatic AFIB/AFL who are currently in sinus rhythm. Sotalol is also indicated for the treatment of life-threatening ventricular tachycardia.

Intravenous sotalol can be administered to achieve steady state concentration faster compared to conventional oral dosing (e.g., 1-3 days for a non-renally impaired patient).

Typically, IV sotalol is diluted for infusion. For example, IV sotalol can be diluted in saline, 5% dextrose in water (D5W), or Ringer's lactate. The dilution volume chosen is one that is convenient for administration and consistent with fluid restriction. A volumetric infusion pump can be used to administer the IV sotalol.

Typically, other antiarrhythmic therapy is withdrawn prior to starting sotalol.

In embodiments, the IV dose is a substitute for an oral dose and is delivered by way of an IV bolus or infusion over a period of about 1-5 hours, such as for 1 hour, 1.5 hours, 2 hours, 2.5 hours, 3 hours, 3.5 hours, 4 hours, 4.5 hours, or about 5 hours, and so on, or within any range with any of these values as lower and upper values. The IV dose can be an initial IV dose or an IV loading dose or an IV maintenance dose. In embodiments, the infusion time and/or the amount of the IV dose is selected based on the creatinine clearance of the subject/patient and when repeated at a selected interval is capable of providing a sotalol concentration commensurate with the steady state concentration expected from multiple sequential oral doses. In embodiments, in response to the administering of the IV dosage, the subject is capable of achieving a peak serum, plasma or blood concentration of sotalol in the range of 90-110% of a Cₘₐₓ at steady-state expected for that subject from oral dosing. In embodiments, the IV dosing protocol is capable of providing a sotalol concentration commensurate with the steady state concentration expected from multiple sequential oral doses of a selected oral dosing regimen.

In embodiments, the IV doses can be administered while the patient/subject is admitted to a hospital or other facility capable of providing cardiac resuscitation and continuous electrocardiographic monitoring. In embodiments, one or more of the IV doses can be administered to the patient/subject after release from a hospital or other facility capable of providing cardiac resuscitation and continuous electrocardiographic monitoring, and electrocardiographic monitoring can be continued if desired by any method or device capable of providing the same or similar monitoring, such as at home or at a physician's office or at a facility capable of providing such monitoring.

In embodiments, the IV dose is administered in an amount of about 60 mg, about 75 mg, or about 90 mg, such as about 55 mg to about 65 mg, about 50 mg to about 70 mg, about 70 mg to about 80 mg, about 65 mg to about 85 mg, about 85 mg to about 95 mg, about 80 mg to about 100 mg, or about 45 mg to about 105 mg, or about 110-128 mg, or about 99-141 mg or about 49.5-70.2 mg, or about 60-75 mg, or about 73.8-105.6 mg, or from about 90-125 mg, such as from about 112.5 to 125 mg, or about 63-88.2 mg, or about 55-64 mg, or about 75-82.5 mg, or about 82-96 mg, or about 49.5-70.4 mg, or about 73.8-105.6 mg, of from about 90-105 mg, or about 63-88 mg, or about 70-80 mg, or any range in between. In embodiments, one or more IV doses can be administered in an amount of 67.5 mg, 75 mg, 105 mg, 112.5 mg, 135 mg, 142.5 mg, or 150 mg. One or more IV dose can be an initial IV dose and/or a subsequent IV dose and can be expressed as mg/min, for example, as a rate of from about 0.825-1.17 mg/min (49.5-70.2 mg), or from about 1.23-1.76 mg/min (73.8-105.6 mg), or from about 1.65-2.35 mg/min (99-140.8 mg), or from about 1.05-1.47 mg/min (66-88.2 mg). In embodiments, any one or more IV dose can be administered at a rate in the range of about 0.13-0.58 mg/min (40-175 mg), such as about 0.225-0.5 mg/min (67.5-150 mg), including at a rate of about 0.16-0.33 mg/min (50-100 mg), or about 0.14-0.3 mg/min (42.5-92.5 mg), or about 0.29-0.45 mg/min (87.5-137.5 mg), or about 0.26-0.43 mg/min (80-130 mg), or in the range of about 0.41-0.58 mg/min (125-175 mg), or about 0.97-0.55 mg/min (117.5-167.5 mg), or in the range of about 0.45-0.475 mg/min (135-142.5 mg), or in the range of about 0.36-0.53 mg/min (110-160 mg), and so on.

In embodiments, one or more IV dose is administered, with a first IV dose being administered immediately or about 1-12 hours after administration or initiation of another IV or oral dose, such as about 1 hour to about 6 hours, or about 2-8 hours, after the start or conclusion of the administration of another IV dose, such as about 1.25 hours, 1.5 hours, 1.75 hours, 2 hours, 2.25 hours, 2.5 hours, 2.75 hours, 3 hours, 3.25 hours, 3.5 hours, 3.75 hours, 4 hours, 4.25 hours, 4.5 hours, 4.75 hours, 5 hours, 5.25 hours, 5.5 hours, 5.75 hours, 6 hours, 8 hours, 12 hours, 16 hours, 18 hours, 20 hours, 24 hours, 30 hours, 36 hours, 40 hours, or 48 hours after the start or conclusion of the administration or initiation of any IV or oral dose. In embodiments, a subsequent IV dose is administered about 1 hour to about 12 hours after the start or conclusion of the administration of an initial IV dose or another IV dose, such as about 2 hours to about 11 hours, about 3 hours to about 10 hours, about 4 hours to about 9 hours, about 5 hours to about 8 hours, or about 6 hours to about 7 hours, or about 12 hours to about 24 hours, or about 24 hours to about 48 hours after the start or conclusion of the administration of an initial IV dose or another IV dose.

In embodiments, the subject/patient is administered a subsequent IV dose after being released from the hospital or other facility that was providing the monitoring, but the patient/subject can still be monitored in a similar way, such as by using a portable/wearable ECG monitoring system, or not monitored in such a way. In other embodiments, the subject/patient is administered a subsequent IV dose while admitted to the hospital or other facility providing the monitoring. In other embodiments, the subject/patient is administered two or more subsequent IV doses while admitted to the hospital or other facility providing the monitoring, such as 2, 3, 4 or more subsequent IV doses.

In embodiments, amounts of the oral doses are selected from about 80 mg, about 120 mg, or about 160 mg, or about 240 mg. In embodiments, more than one oral dose amount is given to a particular patient (for example, a patient receives one or more oral dose at 120 mg and a subsequent higher oral dose of 160 mg or a subsequent lower oral dose of 80 mg, or any combination thereof). In embodiments, one or more IV dose can be provided or administered as a substitute for any one or more oral dose and/or an IV dosing protocol can be substituted for an oral dosing protocol.

One or more IV dose can be intravenously infused into a central or peripheral vein of the patient. The flow rate of the intravenous dose can be adjusted based on the concentration of the sotalol in the intravenous formulation, the desired duration of administration, and the body weight of the patient to achieve a specific dose. Thus, longer administration durations of a specific dose (X μg/kg or mg/kg) will typically have slower flow rates compared to shorter administration durations to achieve the same dose.

In embodiments, the IV dose is selected based on a subject's/patient's creatinine clearance. For example, in some cases a patient/subject with a lower creatinine clearance may receive a lower IV dose than a patient with a higher creatinine clearance, or in some cases a patient/subject with a higher creatinine clearance may receive a lower IV dose than a patient with a lower creatinine clearance. In some cases, for example, the amount of IV sotalol hydrochloride appropriate to administer to a subject with a CrCl of 10-29 mL/min or 30-59 mL/min, can be higher than is appropriate for a subject with a CrCl of ≥90 mL/min.

In embodiments, the amount of IV sotalol hydrochloride administered to a subject with a creatinine clearance in the range of 60-89 mL/min (e.g., mild renal impairment) can be the same as the amount administered to a subject with normal kidney function (e.g., creatinine clearance in the range of ≥90 mL/min), whereby no adjustment in the amount of the dose of sotalol hydrochloride is made to accommodate for the degree of renal impairment, yet the IV dosing protocol is capable of providing a sotalol concentration commensurate with the steady state concentration expected from the multiple sequential oral doses for both such subjects. Alternatively or additionally, no adjustment in the time between doses (e.g., dosing interval) is made to accommodate for the degree of renal impairment, such that a subject with a creatinine clearance in the range of 60-89 mL/min (e.g., mild renal impairment) can be administered the IV dosing protocol with IV dosing occurring at the same timing interval as a subject with normal kidney function (e.g., creatinine clearance in the range of ≥90 mL/min). Such dosing can be administered when the multiple sequential oral doses are 80 mg, 120 mg, 160 mg, or 240 mg.

In embodiments, one or more of the subsequent or additional IV doses is selected based on a patient's change in QTc (for example, a patient that experiences an increase in QTc of less than 20% after receiving an IV dose may receive a higher IV dose than a patient that experiences an increase in QTc of 20% or greater after receiving the IV dose). In various implementations, the change exceeding baseline QTc can be 5%, 10%, 15%, 20%, or 25% over baseline QTc. The QTc can be measured at any point and/or at regular intervals, such as every 10, 15, 20, 30, 45, or 60 minutes during treatment. In some situations, such as if the patient's heart rate is less than 60 bpm, the uncorrected QT interval can be used. Remote ECG monitoring, such as Holter monitoring or event monitoring, may be indicated for long term monitoring of QT interval or QTc. In embodiments, the amount and/or interval of one or more subsequent IV dose is changed after administration of a previous IV dose due to a change in the patient's QTc (for example, for a patient given an IV dose of 175 mg who may then experience an increase in QTc of 20% or greater, the next IV dose can be lowered to an amount in the range of 80-130 mg).

In embodiments, the subject/patient is administered an initial IV dose and multiple subsequent IV doses prior to being released from the hospital or other facility capable of providing cardiac resuscitation and continuous electrocardiographic monitoring. In other embodiments, an initial IV dose and a first subsequent IV dose are administered in a hospital or other facility that can provide cardiac resuscitation and continuous electrocardiographic monitoring and one or more second or subsequent IV doses are administered, such as by the patient according to a prescription, after discharge from the facility/hospital. In embodiments, one IV dose is administered in a hospital or other facility that can provide cardiac resuscitation and continuous electrocardiographic monitoring and a subsequent dose is administered by the patient/subject after discharge from the facility/hospital, but may still be under monitoring, such as self-monitoring.

In embodiments, subsequent IV dose(s) are administered at intervals of about 12 hours, about 24 hours, or about 48 hours from a prior IV dose, such as the most recent previous IV dose.

In embodiments, the IV dose(s) are given in amounts and at time intervals to enable maximum serum concentration of the subject within a range of ±25% of a Cₘₐₓ at steady state predicted for the subject for an oral dosing protocol of 80 mg, 120 mg or 160 mg, or 240 mg sotalol. In embodiments, the dosing protocol is selected to render the subject/patient capable of achieving Cₘₐₓss within 24 hours, such as within 1 hour, of the start of the administration of the IV dose. In embodiments, Cₘₐₓss is achieved before, during and/or after administration of one or more subsequent IV dose(s). In embodiments, as a result of one or more IV doses, the patient experiences or is capable of experiencing a Cₘₐₓ at steady state that is at least about 85% of the Cₘₐₓss, such as about 87%, 90%, 92%, 95%, 97% or 99% of the Cₘₐₓss for the patient's dosing protocol. In embodiments, the IV dosing protocol is capable of providing a sotalol concentration commensurate with the steady state concentration expected from multiple sequential oral doses of a selected oral dosing regimen.

In embodiments, the IV dose comprises an amount of sotalol hydrochloride capable of providing exposure to the drug that is commensurate with the exposure expected from a selected oral dosing regimen being substituted with the IV dosing. For example, if the median maximum concentration at steady state expected from oral dosing of 80 mg every 12 hours is in the range of 820 ng/mL to 860 ng/ml (for example, for a patient with a CrCl in the range of ≥90 mL/min), then the IV dose is selected to provide a similar exposure with a median C_{max_ss} in the range of 820 ng/ml to 860 ng/mL, such as in the range of 840 ng/ml to 850 ng/ml. If the median maximum concentration at steady state expected from oral dosing of 80 mg every 12 hours is in the range of 1200 ng/mL to 1300 ng/ml (for example, for a patient with a CrCL in the range of 60-89 mL/min), then the IV dose is selected to provide a similar exposure with a median C_{max_ss} in the range of 1200 ng/mL to 1300 ng/ml, such as in the range of 1215 ng/ml to 1240 ng/ml. If the median maximum concentration at steady state expected from oral dosing of 120 mg every 24 hours is in the range of 1500 ng/mL to 1700 ng/ml (for example, for a patient with a CrCL in the range of 30-59 mL/min), then the IV dose is selected to provide a similar exposure with a median C_{max_ss} in the range of 1500 ng/ml to 1700 ng/ml, such as in the range of 1590 ng/ml to 1650 ng/mL. If the median maximum concentration at steady state expected from oral dosing of 160 mg every 48 hours is in the range of 2050 ng/ml to 2230 ng/ml (for example, for a patient with a CrCL in the range of 10-29 mL/min), then the IV dose is selected to provide a similar exposure with a median C_{max_ss} in the range of 2050 ng/ml to 2230 ng/mL, such as in the range of 2100 ng/mL to 2200 ng/mL.

In embodiments, the IV dose is commensurate in exposure when providing a sotalol concentration in the subject/patient that is within 5% of the maximum concentration at steady state expected from the oral dosing. In embodiments, when the IV dosing provides a maximum concentration at steady state that is within 1%, 2%, 3%, 4%, 5%, 6%, 7%, 8%, 9%, 10%, 11%, 12%, 13%, 14%, 15%, or 20% of the maximum concentration at steady state expected from oral dosing, the IV dose (or IV dosing protocol) is capable of providing a sotalol concentration in the subject/patient that is commensurate with the steady state concentration expected from multiple sequential oral doses of a selected oral dosing regimen. For example, the IV dose can be selected to provide a median maximum concentration at steady state that is within 5-15% of the maximum concentration at steady state expected from 80 mg oral dosing or 120 mg oral dosing or 160 mg oral dosing or 240 mg oral dosing, or within 5-8% of the maximum concentration at steady state expected from 80 mg, 120 mg, 160 mg or 240 mg oral dosing, or within 4-6% of the maximum concentration at steady state expected from 80 mg, 120 mg, 160 mg or 240 mg oral dosing.

TABLE 1
The median steady-state Cₘₐₓ values observed in clinical trial simulations
and the corresponding IV dose regimens by renal function.

| | Dosing | Oral dose (mg) | | | IV dose (mg) | | | | | |
|---|---|---|---|---|---|---|---|---|---|---|
| Renal | interval | 80 | 120 | 160 | 45 | 52.5 | 60 | 67.5 | 75 | 82.5 |
| function | (hours) | Median Cmax, ss (ng/ml) | | | Median Cmax, ss (ng/ml) | | | | | |
| CRCL ≥ 90 | 12 | 843 | 1265 | 1686 | 507 | 591 | 675 | 760 | 844 | 929 |
| (Normal) | | | | | | | | | | |
| CRCL 60-89 | 12 | 1221 | 1832 | 2443 | 741 | 864 | 988 | 1111 | 1235 | 1358 |
| (Mild) | | | | | | | | | | |
| CRCL 30-59 | 24 | 1079 | 1619 | 2159 | 689 | 804 | 919 | 1034 | 1149 | 1263 |
| (Moderate) | | | | | | | | | | |
| CRCL 10-29 | 48 | 1083 | 1624 | 2165 | 711 | 830 | 949 | 1067 | 1186 | 1304 |
| (Severe) | | | | | | | | | | |

| | IV dose (mg) | | | | | | | | |
|---|---|---|---|---|---|---|---|---|---|
| Renal | 90 | 97.5 | 105 | 112.5 | 120 | 127.5 | 135 | 142.5 | 150 |
| function | Median Cmax, ss (ng/ml) | | | | | | | | |
| CRCL ≥ 90 | 1013 | 1098 | 1182 | 1267 | 1351 | 1435 | 1520 | 1604 | 1689 |
| (Normal) | | | | | | | | | |
| CRCL 60-89 | 1482 | 1605 | 1729 | 1852 | 1975 | 2099 | 2222 | 2346 | 2469 |
| (Mild) | | | | | | | | | |
| CRCL 30-59 | 1378 | 1493 | 1608 | 1723 | 1838 | 1953 | 2067 | 2182 | 2297 |
| (Moderate) | | | | | | | | | |
| CRCL 10-29 | 1423 | 1541 | 1660 | 1778 | 1897 | 2016 | 2134 | 2253 | 2371 |
| (Severe) | | | | | | | | | |

CRCL, creatinine clearance (mL/min), Cmax_ss, maximum concentration at steady state; IV, intravenous

The underlined IV doses provide exposures clinically equivalent to the oral doses. Clinically equivalent in the context of this disclosure refers to being within a range capable of providing the same or a similar effect (e.g., commensurate with) in the subject/patient. For example, a patient with a creatinine clearance in the range of 60-89 mL/min, administered an oral dosing protocol of 120 mg oral sotalol hydrochloride at a 12-hour interval, might be expected to have a median steady-state C_{max_ss} of about 1832 ng/mL. An IV dosing regimen can be selected to replace the oral dosing protocol by selecting an IV dose that would be capable of achieving the sotalol concentration commensurate with the steady state concentration expected from the oral dosing protocol (e.g., multiple sequential oral doses). As shown in Table 1, such IV dose would be in an amount of 112.5 mg, which when repeated at the same 12-hour interval would be expected to achieve a median steady-state C_{max_ss} in the patient of about 1852 ng/ml.

In embodiments, an IV dose for an IV dosing protocol for a patient with renal impairment can be selected to provide an amount of IV sotalol that would be capable of achieving an exposure/concentration in the renally impaired patient that is commensurate with the exposure/concentration expected of a patient with normal renal function receiving a particular oral dosing regimen. For example, for a patient with mild renal impairment who would have been administered an 80 mg BID oral dosing regimen but who will be administered an IV dosing protocol as a substitute/replacement for the oral dosing, the IV dose can be selected to provide the exposure/concentration expected from 80 mg BID dosing in a patient with normal renal function. As shown in Table 1, the exposure/concentration expected from 80 mg BID dosing in a patient with normal renal function is a median steady-state C_{max_ss} of about 843 ng/ml. For a patient with mild renal impairment, a similar exposure would be achieved by administering IV doses comprising an amount of sotalol hydrochloride in the range of about 45-52.5 mg (741-864 ng/ml). Similarly, a patient with moderate renal impairment who is administered 120 mg oral sotalol once daily could instead be administered IV doses once daily in an amount of about 82.5 mg, which would be expected to provide an exposure/concentration (1263 ng/ml) commensurate with that of a patient with normal kidney function who takes 120 mg BID (1265 ng/ml). Any substitution of an oral dose for an IV dose can be made in a similar manner from Table 1, including any range of concentrations and/or any range of IV doses enumerated therein and/or in combination with other values (which can be used as endpoints for ranges as well) provided in this specification.

For example, the IV dosing protocol can comprise at least three IV doses, whereby after administration of the third IV dose the subject is capable of and/or achieves a steady state concentration equivalent and/or commensurate with the steady state concentration expected from multiple sequential oral doses, such as at least 5 oral doses. In embodiments, at least five IV doses can be administered to achieve a steady state concentration equivalent and/or commensurate with the steady state concentration expected from multiple sequential oral doses, such as at least three oral doses. Further, for example, at least 1, 2, 3, 4, 5, 6, 7, 8, 9 or 10 IV dose(s) can be administered to achieve a steady state concentration equivalent and/or commensurate with the steady state concentration expected from multiple sequential oral doses, such as at least two oral doses, or at least three oral doses, or at least four oral doses, or at least five oral doses, or at least six oral doses, and so on.

In embodiments of the invention, IV sotalol can be administered as a substitute for oral dosing. In embodiments, sotalol therapy is administered to a subject/patient in need thereof (e.g., for treating a cardiovascular condition described herein) by way of an IV dosing protocol capable of providing a sotalol concentration commensurate with the steady state concentration expected from multiple sequential oral doses, which IV dosing protocol comprises an amount of a sotalol hydrochloride IV dose to be repeated at a selected interval. In embodiments, the IV doses are administered over a period of about 5 hours. The amount and/or timing of the IV doses is based on a corresponding oral sotalol hydrochloride dosing regimen capable of providing a desired effect in the subject as a result of multiple sequential oral doses (such as 80 mg, 120 mg, or 160 mg, or 240 mg), and based on creatinine clearance of the subject.

In embodiments, IV sotalol may be administered as a substitution for one or more oral dose of sotalol. Situations where IV dosing in lieu of oral dosing (e.g., as a substitute for) may be appropriate include those in which the patient cannot take oral administration (NPO), or for gastrointestinal conditions resulting in poor absorption, recovery from GI surgery, and/or surgery or intensive care. In embodiments of the invention, an IV dose selected from Table 1 or 2 is administered to a patient in need thereof and optionally repeated at a selected interval. The IV doses can be administered over any period of time as described herein, such as over 5 hours.

TABLE 2
Intravenous doses for sotalol infusion when
used as a substitute for oral sotalol

| Creatinine | | | |
|---|---|---|---|
| Clearance* | | | |
| (mL/min) | Substituted | | |
| (renal | Oral Dose | | Dosing |
| function/impairment) | (mg) | IV Dose (mg) | Interval (h) |
| ≥90 (normal) | 80 | 50-100, e.g., 75 | 12 |
| 60-89 (mild) | | 50-100, e.g., 75 | 12 |
| 30-59 (moderate) | | 42.5-92.5, e.g., 67.5 | 12 or 24 |
| 10-29 (severe) | | 42.5-92.5, e.g., 67.5 | 12, 24 or 48 |
| ≥90 (normal) | 120 | 87.5-137.5, e.g., 112.5 | 12 |
| 60-89 (mild) | | 87.5-137.5, e.g., 112.5 | 12 |
| 30-59 (moderate) | | 80-130, e.g., 105 | 12 or 24 |
| 10-29 (severe) | | 80-130, e.g., 105 | 12, 24 or 48 |
| ≥90 (normal) | 160 | 125-175, e.g., 150 | 12 |
| 60-89 (mild) | | 125-175, e.g., 150 | 12 |
| 30-59 (moderate) | | 117.5-167.5, e.g., 142.5 | 12 or 24 |
| 10-29 (severe) | | 110-160, e.g., 135 | 12, 24 or 48 |

*Calculated using Cockcroft-Gault formula

The Cockcroft-Gault formulas for creatinine clearance (CrCl) are:

$$CrCl ( male ) = ( ( 140 - age ) \times weight in kg ) / ( serum creatinine \times 72 )$$

$$CrCl ( female ) = CrCl ( male ) \times 0.85$$

In embodiments of the invention, the IV dose administered as a substitute for oral sotalol is infused over a period of about 5 hours. In embodiments, the infusion is administered over about 1, 2, 3, 4, 5, 6, 7, 8, 9, or 10 hours.

The IV dose of sotalol, if given, depends on the target concentration and/or creatinine clearance/renal function/renal impairment of the patient/subject and/or the sotalol concentration commensurate with the steady state concentration expected from the multiple sequential oral doses for which the IV dosing is substituting.

Table 2 shows that the IV doses for substitution of a target oral dose of 80 mg are in the range of about 50-100 mg (for both ≥90 mL/min CrCl and 60-89 mL/min CrCl) and in the range of about 42.5-92.5 mg (for both 30-59 mL/min CrCl and 10-29 mL/min CrCl). Additional examples of IV doses for the substitution of an oral target dose of 80 mg include about 55, 56, 57, 58, 59, 60, 61, 62, 63, 64, 65, 66, 67, 67.5, 68, 69, 70, 71, 72, 73, 74, 75, 76, 77, 78, 79, 80, 81, 82, 83, 84, 85, 86, 87, 88, 89, 90, 91, 92, 93, 94, or 95 mg.

Table 2 shows that the IV doses for substitution of a target oral dose of 120 mg are in the range of about 87.5-137.5 mg (for both ≥90 mL/min CrCl and 60-89 mL/min CrCl) and in the range of about 80-130 mg (for both 30-59 mL/min CrCl and 10-29 mL/min CrCl). Additional examples of IV doses for the substitution of an oral target dose of 120 mg include about 90, 91, 92, 93, 94, 95, 96, 97, 98, 99, 100, 101, 102, 103, 104, 105, 106, 107, 108, 109, 110, 111, 112, 112.5, 113, 114, 115, 116, 117, 118, 119, 120, 121, 122, 123, 124, 125, 126, 127, 128, 129, 130, 131, 132, 133, 134, or 135 mg.

Table 2 shows that the IV doses for substitution of a target oral dose of 160 mg are in the range of about 125-175 mg (for both ≥90 mL/min CrCl and 60-89 mL/min CrCl), and in the range of about 117.5-167.5 (for 30-59 mL/min CrCl), and in the range of about 110-160 mg (for 10-29 mL/min CrCl). Additional examples of IV doses for the substitution of an oral target dose of 160 mg include 130, 131, 132, 133, 134, 135, 136, 137, 138, 139, 140, 141, 142, 142.5, 143, 144, 145, 146, 147, 148, 149, 150, 151, 152, 153, 154, 155, 156, 157, 158, 159, 160, 161, 162, 163, 164, 165, 166, 167, 168, 169, or 170 mg.

In embodiments, a patient/subject can receive an IV dosing protocol capable of mimicking the effect of oral dosing at doses of 240 mg. For example, the dosing amount of the IV doses of the protocol can be in the range of 130 mg to 260 mg, such as 220-240 mg or 210-265 mg (≥90 mL/min CrCl), and 130 mg to 220 mg (60-89 mL/min CrCl, 30-59 mL/min CrCl, and 10-29 mL/min CrCl). Additional examples of the IV dosing to mimic the effect of oral dosing at a target of 240 mg include 125-180 mg, 135-200 mg, or 160-220 mg. Further examples include 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, or 260 mg.

The recommended starting dose of 80 mg is the FDA recommended dosage. A physician can elect to start a patient on a higher dose (e.g., 120 mg, 160 mg, 240 mg), if deemed appropriate, such as accounting for the cardiovascular condition of the subject/patient.

The IV dosing interval refers to the time between one or more of the IV doses. 12 h is B.I.D. (or BID). 24 h is Q.D. (or QD).

In embodiments, sotalol hydrochloride is administered according to any combination of the above or below IV dose amounts, durations and/or intervals to treat or prevent a cardiovascular condition selected from atrial flutter (AFL), atrial fibrillation (AF), persistent atrial fibrillation, paroxysmal atrial fibrillation, treating paroxysmal or persistent atrial fibrillation or atrial flutter, such as with patients who have experienced a recent AF/AFL episode who are in sinus rhythm or who will be cardioverted, treating ventricular fibrillation (VF) or ventricular arrhythmias, treating life-threatening recurrent VF or life-threatening recurrent hemodynamically unstable VT, treating focal atrial tachycardia, supraventricular tachycardia (SVT), atrioventricular re-entrant tachycardia (AVRT), atrioventricular nodal re-entrant tachycardia (AVNRT), atrial tachycardia, paroxysmal atrial tachycardia, junctional tachycardia, junctional ectopic tachycardia (JET), congenital heart disease (CHD), multifocal atrial tachycardia, atrial ectopic tachycardia, accessory pathway SVT, infantile SVT, post-operative SVT, paroxysmal SVT, treating Wolff-Parkinson-White syndrome, ventricular tachycardia (VT), premature ventricular contractions (PVCs), hemodynamically stable or unstable ventricular tachycardia, heart failure, coronary artery disease, and pulmonary artery hypertension, maintenance of normal sinus rhythm (for example, delay in time to recurrence of atrial fibrillation/atrial flutter (AF/AFL)) for example in patients with symptomatic AF/AFL and/or who are currently in sinus rhythm, and/or indicated for the treatment of life-threatening ventricular tachycardia; and/or treating people, such as hospitalized people, with atrial fibrillation cardioverted to normal sinus rhythm, without ventricular arrhythmias or various forms of blocks, in patients with normal kidney function. In embodiments, sotalol can be administered to a patient/subject who is receiving extracorporeal membrane oxygenation (ECMO) and/or for situations where the subject/patient is unable to take an anti-arrhythmic by mouth (NPO). In embodiments, the subject/patient is currently in sinus rhythm, or is not currently in sinus rhythm, or has been or will be converted to sinus rhythm with sotalol or another antiarrhythmic and/or cardioversion, such as electric cardioversion (e.g., DCCV or direct current cardioversion).

The specific intravenous dose and rate protocols can be selected based upon one or more of the patient's condition, baseline and in-treatment QT interval or QTc, and/or the patient's creatinine clearance as described herein. Other factors affecting selection of the IV dosing protocol include patient body weight. Oral doses of sotalol hydrochloride for which IV doses can be substituted include, for example 80 mg, 120 mg, 160 mg or 240 mg, and the like. Situations where IV doses are appropriate include those in which the patient cannot take oral administration (NPO), or for gastrointestinal conditions resulting in poor absorption, recovery from GI surgery, and/or intensive care. Sotalol administration according to embodiments of the invention is administered to patients naïve to sotalol treatment and/or to patients currently receiving sotalol therapy and/or to patients having previously received sotalol. In embodiments, the sotalol dose is determined by patient characteristics including body weight, sex, and/or creatinine clearance.

In embodiments, the patient receiving sotalol hydrochloride has experienced AF, but is in normal sinus rhythm prior to administration of the sotalol hydrochloride dose. In embodiments, the patient receiving sotalol hydrochloride has experienced AF and is not in normal sinus rhythm prior to administration of the sotalol hydrochloride dose.

According to embodiments, the IV dosing parameters are selected to enable maximum serum concentration at steady state of sotalol in the subject/patient within 24 hours, such as within 1 hour, of the start of the IV administering and within a range of ±25% of a Cₘₐₓ at steady state predicted for the subject for a corresponding oral dosing protocol of 80 mg, 120 mg or 160 mg or 240 mg sotalol. Once it is determined that the patient/subject is capable of tolerating the sotalol by way of the IV dose, subsequent IV dosing begins (e.g., is repeated) before or after the patient/subject has been released from the hospital or other facility providing monitoring. In embodiments, a first subsequent IV dose is administered before the patient/subject is released from the hospital or other facility that was providing the monitoring. If released, the patient/subject can still be monitored in a similar way, such as by using a portable/wearable ECG monitoring system.

The Examples below illustrate various protocols for administering IV sotalol.

### Example 1

In an embodiment of the invention, sotalol therapy is provided by administering to a subject/patient in need thereof an IV dose of sotalol hydrochloride, which when based on the subject's creatinine clearance and repeated at a selected interval is capable of providing the subject with a sotalol concentration commensurate with the steady state concentration expected from a selected oral dosing regimen (e.g., multiple sequential oral doses). In embodiments, the IV dosing (amount and/or interval) is based on the sotalol concentration expected from a selected number of oral doses, such as an equivalent number of oral doses. The IV dosing can have the same or a different number (more doses or fewer doses) of doses as the complementary oral dosing regimen. Exemplary amounts for the IV doses are in Table 1 or Table 2.

The IV doses are delivered via IV infusion over a period of 1 hour, 1.5 hours, 2 hours, 2.5 hours, 3 hours, 3.5 hours, 4 hours, 4.5 hours, or 5 hours (or any range within these values as endpoints of the range) and is administered/delivered to the patient/subject while admitted to a hospital or other facility capable of providing cardiac resuscitation and continuous electrocardiographic monitoring.

Once it is determined that the patient/subject is capable of tolerating the sotalol by way of the IV dose, subsequent IV dosing begins before or after the patient/subject has been released from the hospital or other facility providing monitoring. In embodiments, at least one IV dose is administered before the patient/subject is released from the hospital or other facility that was providing the monitoring. If released, the patient/subject can still be monitored in a similar way, such as by using a portable/wearable ECG monitoring system and/or monitored in the same or a similar facility.

In embodiments, the interval for repeating the IV dosing depends on the CrCl of the patient. For IV doses given over a period of 5 hours and for CrCl of ≥90 mL/min and 60-89 mL/min, the IV dosing interval is a 12-hour interval. For CrCl of 30-59 mL/min, the IV dosing interval is a 12-hour or 24-hour interval, and for CrCl of 10-29 mL/min, the IV dosing interval is a 12-hour, 24-hour or 48-hour interval.

### Example 2

An example sotalol treatment protocol for a patient experiencing AF and who is currently in normal sinus rhythm (which patient could have resumed normal sinus rhythm spontaneously, or by way of chemical cardioversion, or electrocardioversion) is described herein. The male patient, age 60, is admitted to a hospital or other facility capable of providing cardiac resuscitation and continuous electrocardiographic monitoring. The patient's creatinine clearance is determined to be 60-89 mL/min (mild renal impairment). The patient is connected to an electrocardiographic and an initial QTc is determined to be less than 450 ms. If the patient were to be administered multiple sequential oral doses, the physician would have selected an oral sotalol dosing regimen of 80 mg at a 12-hour interval. However, due to the patient's NPO status, the physician determines the patient should instead be administered an IV sotalol dosing regimen as a substitute for the selected oral dosing regimen. IV doses in an amount of 75 mg sotalol hydrochloride are administered to the patient at 12-hour interval as a substitute for oral dosing. It is noted that notwithstanding the patient's mild kidney impairment, no adjustment to the IV dosing amount or interval is made. That is, even though the patient's CrCl is lower than normal, neither the dose is adjusted lower nor the dosing interval increased to accommodate for the mild renal impairment. The 75 mg IV dose is administered to the patient over a period of 5 hours. The patient's QTc interval is measured, along with heart rate and blood pressure at selected times to ensure that the patient is tolerating the drug. The patient's QTc interval does not exceed 500 ms during any of these measurements, and the ΔQTc is less than 20%. Once it is determined that the patient/subject is capable of tolerating the sotalol, one or more subsequent doses of sotalol hydrochloride in an amount of 75 mg is administered to the subject at a 12-hour interval. The patient's QTc interval can continue to be monitored. The patient's QTc interval still does not exceed 500 ms and the patient's ΔQTc is less than 20%.

The patient would or is expected to achieve a Cₘₐₓ at steady state from one or multiple sequential IV doses, such as within 1, 2, 3, 4, 5, or 6 hours, or within 7, 8, 9, 10, 11, 12, 13, 14, 15, 16, 17, 18, 19, 20, 21, 22, 23, or 24 hours of starting the IV dosing.

### Example 3

An example anti-arrhythmic/sotalol IV treatment protocol is described herein for a patient with paroxysmal SVT who is currently in normal sinus rhythm. The female patient, who had been taking 160 mg oral sotalol once per day, is admitted to the ICU and intubated. A physician determines the patient should receive IV sotalol as a substitute for her 160 mg oral dosing regimen. The patient's creatinine clearance is determined to be in the range of 30-59 mL/min. IV sotalol doses in an amount of 142.5 mg are administered to the patient via 5 hour IV infusions at a 24-hour interval. The IV dosing is continued until the patient/subject is able to resume the 160 mg oral dosing regimen.

### Example 4

An example anti-arrhythmic/sotalol treatment protocol is described herein for a patient with a cardiovascular condition and who is in normal sinus rhythm. The male patient, age 60 and previously taking 120 mg oral sotalol twice per day, is admitted for treatment in a hospital and is NPO. A physician determines the patient should receive IV sotalol doses as substitute for the 120 mg oral dosing regimen of sotalol; however, the patient's creatinine clearance, which was previously normal, is determined to be 30-59 mL/min upon hospital admission. The patient is therefore administered 105 mg IV sotalol over a period of about 5 hours and once per day.

### Example 5

An example sotalol treatment protocol for a patient experiencing VT who is not currently in normal sinus rhythm is described herein. The female patient, age 75, is admitted to a hospital or other facility capable of providing cardiac resuscitation and continuous electrocardiographic monitoring. The patient's creatinine clearance is determined to be 10-29 mL/min (severe renal impairment). The patient is connected to an electrocardiographic and an initial QTc is determined to be less than 450 ms. If the patient were to be administered multiple sequential oral doses, the physician would have selected an oral sotalol dosing regimen of 120 mg at a 48-hour interval. However, the physician determines the patient should instead be administered an IV sotalol dosing regimen as a substitute for the selected oral dosing regimen. IV doses in an amount of 105 mg sotalol hydrochloride are administered to the patient at 48-hour interval as a substitute for oral dosing. The 105 mg IV dose is administered to the patient over a period of 5 hours. The patient's QTc interval is measured, along with heart rate and blood pressure at selected times to ensure that the patient is tolerating the drug. The patient's QTc interval does not exceed 500 ms during any of these measurements, and the ΔQTc is less than 20%. Once it is determined that the patient/subject is capable of tolerating the sotalol, one or more subsequent doses of sotalol hydrochloride in an amount of 105 mg is administered to the subject at a 48-hour interval. The patient's QTc interval can continue to be monitored. The patient's QTc interval still does not exceed 500 ms and the patient's ΔQTc is less than 20%.

### Example 6

An example sotalol treatment protocol for a patient with atrial flutter (AFL) and persistent atrial fibrillation who is not currently in normal sinus rhythm is described herein. The male patient, age 65, is admitted to a hospital or other facility capable of providing cardiac resuscitation and continuous electrocardiographic monitoring. The patient's creatinine clearance is determined to be ≥90 mL/min. The patient is connected to an electrocardiographic and an initial QTc is determined to be less than 450 ms. If the patient were to be administered multiple sequential oral doses, the physician would have selected an oral sotalol dosing regimen of 160 mg at a 12-hour interval. However, the physician determines the patient should instead be administered an IV sotalol dosing regimen as a substitute for the selected oral dosing regimen. IV doses in an amount of 150 mg sotalol hydrochloride are administered to the patient at 12-hour interval as a substitute for oral dosing. The 150 mg IV dose is administered to the patient over a period of 5 hours. The patient's QTc interval is measured, along with heart rate and blood pressure at selected times to ensure that the patient is tolerating the drug. The patient's QTc interval does not exceed 500 ms during any of these measurements, and the ΔQTc is less than 20%. Once it is determined that the patient/subject is capable of tolerating the sotalol, one or more subsequent doses of sotalol hydrochloride in an amount of 150 mg is administered to the subject at a 12-hour interval. The patient's QTc interval can continue to be monitored. The patient's QTc interval still does not exceed 500 ms and the patient's ΔQTc is less than 20%.

The patient would or is expected to achieve a Cₘₐₓ at steady state from one or multiple sequential IV doses, such as within 1, 2, 3, 4, 5, or 6 hours, or within 7, 8, 9, 10, 11, 12, 13, 14, 15, 16, 17, 18, 19, 20, 21, 22, 23, or 24 hours of starting the IV dosing.

### Example 7

An example sotalol treatment protocol for a patient with premature ventricular contractions (PVCs) who is currently in normal sinus rhythm is described herein. The female patient, age 55, is admitted to a hospital or other facility capable of providing cardiac resuscitation and continuous electrocardiographic monitoring. The patient's creatinine clearance is determined to be in the range of 60-89 mL/min. The patient is connected to an electrocardiographicmd an initial QTc is determined to be less than 450 ms. If the patient were to be administered multiple sequential oral doses, the physician would have selected an oral sotalol dosing regimen of 120 mg at a 12-hour interval. However, the physician determines the patient should instead be administered an IV sotalol dosing regimen as a substitute for the selected oral dosing regimen. IV doses in an amount of 112.5 mg sotalol hydrochloride are administered to the patient at 12-hour interval as a substitute for oral dosing. The 112.5 mg IV doses are administered to the patient for a duration of 5 hours. The patient's QTc interval is measured, along with heart rate and blood pressure at selected times to ensure that the patient is tolerating the drug. The patient's QTc interval does not exceed 500 ms during any of these measurements, and the ΔQTc is less than 20%. Once it is determined that the patient/subject is capable of tolerating the sotalol by IV, one or more subsequent doses of IV sotalol hydrochloride in an amount of 150 mg (to escalate the patient to a higher sotalol concentration) are administered to the subject at a 12-hour interval from a subsequent dose. The patient's QTc interval can continue to be monitored. The patient's QTc interval still does not exceed 500 ms and the patient's ΔQTc is less than 20%.

The patient would or is expected to achieve the desired Cₘₐₓ at steady state from one or multiple sequential IV doses, such as within 1, 2, 3, 4, 5, or 6 hours, or within 7, 8, 9, 10, 11, 12, 13, 14, 15, 16, 17, 18, 19, 20, 21, 22, 23, or 24 hours of starting the IV dosing.

### Example 8

An example anti-arrhythmic/sotalol treatment protocol is described herein for a patient with a cardiovascular condition, whether the patient is or is not in normal sinus rhythm. It has been determined that the female patient, age 45, who has severe renal impairment, should be administered sotalol hydrochloride. The physician determines that an appropriate oral sotalol regimen would be 160 mg taken at a 48-hour interval. It is determined, however, to substitute the oral regimen instead with an IV sotalol hydrochloride treatment protocol. With the goal of providing the patient with an exposure/concentration of sotalol commensurate with what a patient with normal renal function would experience by taking the same protocol, the patient is administered an IV protocol comprising 5-hour infusions of sotalol hydrochloride in an amount in the range of about 97.5-112.5 mg, such as 105 mg, at a 48-hour interval. This is determined to provide the patient with a median steady-state Cₘₐₓ in the range of about 1541-1778 ng/ml after a number of sequential IV doses, which is commensurate with the exposure/concentration that would be expected from a patient with normal renal function being administered the same oral dosing protocol, or about 1686 ng/mL.

### Example 9

An example anti-arrhythmic/sotalol treatment protocol is described herein for a patient with a cardiovascular condition. The patient can be any age and can be currently in sinus rhythm, or not currently in sinus rhythm, or the patient was or will be converted to sinus rhythm with sotalol or another antiarrhythmic and/or cardioversion technique, such as electric cardioversion (e.g., DCCV or direct current cardioversion). The cardiovascular condition can be any one or more of atrial flutter (AFL), atrial fibrillation (AF), persistent atrial fibrillation, paroxysmal atrial fibrillation, paroxysmal or persistent atrial fibrillation or atrial flutter, such as for patients who have experienced a recent AF/AFL episode who are in sinus rhythm or who will be cardioverted, ventricular fibrillation (VF) or ventricular arrhythmias, life-threatening recurrent VF or life-threatening recurrent hemodynamically unstable VT, focal atrial tachycardia, supraventricular tachycardia (SVT), atrioventricular re-entrant tachycardia (AVRT), atrioventricular nodal re-entrant tachycardia (AVNRT), atrial tachycardia, paroxysmal atrial tachycardia, junctional tachycardia, junctional ectopic tachycardia (JET), congenital heart disease (CHD), multifocal atrial tachycardia, atrial ectopic tachycardia, accessory pathway SVT, infantile SVT, post-operative SVT, paroxysmal SVT, treating Wolff-Parkinson-White syndrome, ventricular tachycardia (VT), premature ventricular contractions (PVCs), hemodynamically stable or unstable ventricular tachycardia, heart failure, coronary artery disease, and pulmonary artery hypertension, maintenance of normal sinus rhythm (for example, delay in time to recurrence of atrial fibrillation/atrial flutter (AF/AFL)) for example in patients with symptomatic AF/AFL and/or who are currently in sinus rhythm, and/or indicated for the treatment of life-threatening ventricular tachycardia; and/or treating people, such as hospitalized people, with atrial fibrillation cardioverted to normal sinus rhythm, without ventricular arrhythmias or various forms of blocks, in patients with normal kidney function. In embodiments, sotalol can be administered to a patient who is receiving extracorporeal membrane oxygenation (ECMO).

The physician determines the desired oral sotalol therapy for the particular patient and selects an IV protocol that will provide (by way of a substitute for the oral dosing) a sotalol exposure clinically equivalent to the oral doses and/or oral dosing protocol. The IV doses (amount, duration and/or frequency) can be selected based on the effect that an oral dosing protocol would have on a similar patient with renal impairment and/or can be based on the effect that an oral dosing protocol would have on a patient with a normal creatinine clearance. The IV doses can be selected from the doses provided in either Table 1 and/or Table 2 (above).

The IV dose(s) are administered as a substitute for oral sotalol and are infused over a period of about 5 hours at a selected interval, typically based on the patient's renal function. The IV dosing protocol can comprise any number of IV doses that can be administered to the patient as a substitution for oral doses of 80 mg, 120 mg, 160 mg, or 240 mg.

As substation for an 80 mg oral dosing protocol, the amount of the sotalol IV dose is in the range of about 50-100 mg (for both ≥90 mL/min CrCl and 60-89 mL/min CrCl) and in the range of about 42.5-92.5 mg (for both 30-59 mL/min CrCl and 10-29 mL/min CrCl) (see Table 2). Alternatively or in addition, Table 1 shows that the IV doses for substitution of a target oral dose of 80 mg can be in the range of about 60-90 mg (at a 12-hour interval) for a patient with normal renal function, which IV dosing is determined to provide the patient with a median steady-state Cₘₐₓ in the range of about 675-1013 ng/ml and which is commensurate with the exposure/concentration that would be expected from 80 mg oral dosing in a patient with normal renal function. If the patient has mild renal impairment, IV dosing at an amount in the range of about 60-90 mg (also at a 12-hour interval) is determined to provide the patient with a median steady-state Cₘₐₓ in the range of about 988-1482 ng/ml and which is commensurate with the exposure/concentration that would be expected from 80 mg oral dosing in a patient with mild renal impairment. For moderate renal impairment, the patient could be administered an amount of IV sotalol in the range of about 52.5-82.5 mg (at a 24-hour interval), which is determined to provide the patient with a median steady-state Cₘₐₓ in the range of about 804-1236 ng/ml and which is commensurate with the exposure/concentration that would be expected from 80 mg oral dosing in a patient with moderate renal impairment. For severe renal impairment, a patient could be administered an amount of IV sotalol in the range of about 52.5-82.5 mg (at a 48-hour interval), which is determined to provide the patient with a median steady-state Cₘₐₓ in the range of about 830-1304 ng/mL and which is commensurate with the exposure/concentration that would be expected from 80 mg oral dosing in a patient with severe renal impairment. For a patient with renal impairment, if trying to match the exposure that a patient with normal creatinine clearance would have while taking 80 mg oral doses, the patient can be administered an amount of IV sotalol in the range of about 45-60 mg by IV, which can be administered at an interval of 12 hours (for mild renal impairment) or 24 hours (for moderate renal impairment) or 48 hours (for severe renal impairment).

Table 2 shows that the IV doses for substitution of a target oral dose of 120 mg are in the range of about 87.5-137.5 mg (for both ≥90 mL/min CrCl and 60-89 mL/min CrCl) and in the range of about 80-130 mg (for both 30-59 mL/min CrCl and 10-29 mL/min CrCl). Alternatively, or in addition, Table 1 shows that the IV doses for substitution of a target oral dose of 120 mg can be in the range of about 97.5-127.5 mg for normal and mild renal impairment or in the range of about 90-120 mg for moderate or severe renal impairment. For a patient with renal impairment, if trying to match the exposure that a patient with normal creatinine clearance would have while taking 120 mg oral doses, the patient can be administered IV sotalol dose(s) in an amount in the range of about 75-82.5 mg at a 12-hour interval (for mild renal impairment), or an amount in the range of about 75-90 mg by IV at a 24-hour interval (for moderate renal impairment) or at a 48-hour interval (for severe renal impairment).

Table 2 shows that the IV doses for substitution of a target oral dose of 160 mg are in the range of about 125-175 mg (for both ≥90 ml/min CrCl and 60-89 mL/min CrCl), and in the range of about 117.5-167.5 (for 30-59 mL/min CrCl), and in the range of about 110-160 mg (for 10-29 mL/min CrCl). Alternatively, or in addition, Table 1 shows that the IV doses for substitution of a target oral dose of 160 mg can be in the range of about 142.5-160 mg for normal creatinine clearance or mild renal impairment, or in the range of about 135-150 mg at a 24-hour interval for moderate renal impairment, or in the range of about 127.5-142.5 mg at a 48-hour interval for severe renal impairment. For a patient with renal impairment, if trying to match the exposure that a patient with normal creatinine clearance would have while taking 160 mg oral doses, the patient can be administered an amount in the range of about 90-112.5 mg by IV at a 12-hour interval (for mild renal impairment), or an amount in the range of about 97.5-112.5 mg by IV at a 24-hour interval (for moderate renal impairment) or at a 48-hour interval for severe renal impairment.

If 240 mg oral dosing is determined for the patient, an IV dosing protocol can be administered to the patient that is capable of mimicking the effect of oral dosing at doses of 240 mg. For example, the dosing amount of the IV doses can be in the range of 130 mg to 260 mg (at a 12-hour, 24-hour or 48-hour interval), such as 220-240 mg or 210-265 mg (≥90 mL/min CrCl), and 130 mg to 220 mg (60-89 mL/min CrCl, 30-59 mL/min CrCl, and 10-29 mL/min CrCl). Additional examples of the IV dosing to mimic the effect of oral dosing at a target of 240 mg include 125-180 mg, 135-200 mg, or 160-220 mg. Further examples include 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, or 260 mg.

Once it is determined that the patient/subject is capable of tolerating the sotalol (e.g., the patient's QTc is within an acceptable range), one or more subsequent doses of IV sotalol hydrochloride can be administered to the patient in accordance with the IV protocol selected. One or more IV doses can be administered to the patient in a hospital or other facility capable of monitoring QTc and one or more subsequent IV doses can be administered to the patient after being released from the hospital/facility and/or the patient can return to the facility to be so monitored and/or can go to another facility for such monitoring.

The present disclosure has described particular implementations having various features. In light of the disclosure provided herein, it will be apparent to those skilled in the art that various modifications and variations can be made without departing from the scope or spirit of the disclosure. One skilled in the art will recognize that the disclosed features may be used singularly, in any combination, or omitted based on the requirements and specifications of a given application or design. When an implementation refers to “comprising” certain features, it is to be understood that the implementations can alternatively “consist of” or “consist essentially of” any one or more of the features. Other implementations will be apparent to those skilled in the art from consideration of the specification and practice of the disclosure.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the disclosure fall within the scope of the disclosure. Further, all of the references cited in this disclosure including patents, published applications, and non-patent literature are each individually incorporated by reference herein in their entireties and as such are intended to provide an efficient way of supplementing the enabling disclosure as well as provide background detailing the level of ordinary skill in the art.

1. A method of intravenously administering sotalol, comprising:
determining a creatinine clearance of a subject;
selecting an oral sotalol hydrochloride dosing regimen capable of providing a desired effect in the subject as a result of multiple sequential oral doses;
selecting an IV dose comprising an amount of sotalol hydrochloride that when based on the subject's creatinine clearance and repeated at a selected interval is capable of providing a sotalol concentration commensurate with the steady state concentration expected from the multiple sequential oral doses; and
for treatment of a cardiovascular condition and as a substitute for the oral dosing regimen, administering to the subject the IV dose repeated at the selected interval to achieve the sotalol concentration commensurate with the steady state concentration expected from the multiple sequential oral doses.

2. A method of substituting oral sotalol with intravenous sotalol hydrochloride, comprising:
a. inputting a subject's creatinine clearance into a treatment management application;
b. inputting into the treatment management application an oral sotalol hydrochloride dosing regimen capable of providing a desired effect when administered to the subject as multiple sequential oral doses;
c. the treatment management application providing an output comprising an IV dose comprising an amount of sotalol hydrochloride that when based on the subject's creatinine clearance and repeated at a selected interval is capable of providing a sotalol concentration commensurate with the steady state concentration expected from the multiple sequential oral doses; and
d. for treatment of a cardiovascular condition and as a substitute for the oral dosing regimen, administering to the subject the IV dose repeated at the selected interval to achieve the sotalol concentration commensurate with the steady state concentration expected from the multiple sequential oral doses.

3. The method of claim 1, wherein:
the amount of each sotalol hydrochloride IV dose for a first subject with mild renal impairment and a creatinine clearance in the range of 60-89 mL/min is the same as for a second subject with normal renal function and a creatinine clearance in the range of ≥90 mL/min; and
the selected dosing interval is the same for the first and second subjects;
whereby no adjustment as to IV dosing amount or interval is needed to accommodate the subject with mild renal impairment relative to the subject with normal renal function in order to achieve the sotalol concentration commensurate with the steady state concentration associated with and expected from the multiple sequential oral doses;
optionally wherein the oral sotalol hydrochloride dosing regimen comprises oral doses in the amount of 80 mg, or 120 mg, or 160 mg.

4. The method of claim 1, wherein the IV dosing protocol is selected from any one or more of the following:

| Creatinine | Current/ | | Dosing |
|---|---|---|---|
| Clearance | Substituted | | Interval |
| (mL/min) | Oral Dose (mg) | IV Dose (mg) | (h) |
| ≥90 | 80 | 50-100, e.g., 75 | 12 |
| 60-89 | | 50-100, e.g., 75 | 12 |
| 30-59 | | 42.5-92.5, e.g., 67.5 | 12 or 24 |
| 10-29 | | 42.5-92.5, e.g., 67.5 | 12, 24 or 48 |
| ≥90 | 120 | 87.5-137.5, e.g., | 12 |
| | | 112.5 | |
| 60-89 | | 87.5-137.5, e.g., | 12 |
| | | 112.5 | |
| 30-59 | | 80-130, e.g., 105 | 12 or 24 |
| 10-29 | | 80-130, e.g., 105 | 12, 24 or 48 |
| ≥90 | 160 | 125-175, e.g., 150 | 12 |
| 60-89 | | 125-175, e.g., 150 | 12 |
| 30-59 | | 117.5-167.5, e.g., | 12 or 24 |
| | | 142.5 | |
| 10-29 | | 110-160, e.g., 135 | 12, 24 or 48. |

5. The method of claim 1, wherein the subject/patient is currently in sinus rhythm, or is not currently in sinus rhythm, or has been or will be converted to sinus rhythm with sotalol or another antiarrhythmic and/or cardioversion, such as electric cardioversion (e.g., DCCV or direct current cardioversion), and sotalol hydrochloride is administered for the treatment of one or more cardiovascular condition selected from atrial flutter (AFL), atrial fibrillation (AF), persistent atrial fibrillation, paroxysmal atrial fibrillation, paroxysmal or persistent atrial fibrillation or atrial flutter, such as for patients who have experienced a recent AF/AFL episode who are in sinus rhythm or who will be cardioverted, ventricular fibrillation (VF) or ventricular arrhythmias, life-threatening recurrent VF or life-threatening recurrent hemodynamically unstable VT, focal atrial tachycardia, supraventricular tachycardia (SVT), atrioventricular re-entrant tachycardia (AVRT), atrioventricular nodal re-entrant tachycardia (AVNRT), atrial tachycardia, paroxysmal atrial tachycardia, junctional tachycardia, junctional ectopic tachycardia (JET), congenital heart disease (CHD), multifocal atrial tachycardia, atrial ectopic tachycardia, accessory pathway SVT, infantile SVT, post-operative SVT, paroxysmal SVT, treating Wolff-Parkinson-White syndrome, ventricular tachycardia (VT), premature ventricular contractions (PVCs), hemodynamically stable or unstable ventricular tachycardia, heart failure, coronary artery disease, and pulmonary artery hypertension, maintenance of normal sinus rhythm (for example, delay in time to recurrence of atrial fibrillation/atrial flutter (AF/AFL)) for example in patients with symptomatic AF/AFL and/or who are currently in sinus rhythm, and/or indicated for the treatment of life-threatening ventricular tachycardia; and/or treating people, such as hospitalized people, with atrial fibrillation cardioverted to normal sinus rhythm, without ventricular arrhythmias or various forms of blocks, in patients with normal kidney function.

6. The method of claim 5, wherein the subject is currently in sinus rhythm and/or has experienced cardioversion to sinus rhythm, for example by way of electrocardioversion, such as DCCV, or chemical cardioversion, such as with sotalol hydrochloride, or combinations thereof.

7. The method of claim 5, wherein:
the oral sotalol hydrochloride dosing regimen comprises 80 mg oral doses;
the creatinine clearance of the subject is in the range of 10-59 mL/min;
the IV dose is administered in an amount of 67.5 mg.

8. The method of claim 5, wherein:
the oral sotalol hydrochloride dosing regimen comprises 120 mg oral doses;
the creatinine clearance of the subject is in the range of 10-59 mL/min;
the IV dose is administered in an amount of 105 mg.

9. The method of claim 5, wherein:
the oral sotalol hydrochloride dosing regimen comprises 160 mg oral doses;
the creatinine clearance of the subject is in the range of 30-59 mL/min;
the IV dosage is administered in an amount of 142.5 mg.

10. The method of claim 5, wherein:
the oral sotalol hydrochloride dosing regimen comprises 160 mg oral doses;
the creatinine clearance of the subject is in the range of 10-29 mL/min;
the IV dose is administered in an amount of 135 mg.

11. The method of claim 1, wherein the selected interval is a 12-hour, 24-hour or 48-hour interval.

12. The method of claim 1, wherein the IV dose is a 5-hour intravenous dose.

13. The method of claim 1, wherein the sotalol concentration is commensurate with the steady state concentration expected from the multiple sequential oral doses when the sotalol concentration is in the range of ±5% or ±10% or ±15% or ±20% of the expected steady state concentration.

14. The method of claim 2, wherein the sotalol concentration is commensurate with the steady state concentration expected from the multiple sequential oral doses when the sotalol concentration is in the range of ±5% or ±10% or ±15% or ±20% of the expected steady state concentration.

15. The method of claim 2, wherein the IV dosing protocol is selected from any one or more of the following:

| Creatinine | Current/ | | Dosing |
|---|---|---|---|
| Clearance | Substituted | | Interval |
| (mL/min) | Oral Dose (mg) | IV Dose (mg) | (h) |
| ≥90 | 80 | 50-100, e.g., 75 | 12 |
| 60-89 | | 50-100, e.g., 75 | 12 |
| 30-59 | | 42.5-92.5, e.g., 67.5 | 12 or 24 |
| 10-29 | | 42.5-92.5, e.g., 67.5 | 12, 24 or 48 |
| ≥90 | 120 | 87.5-137.5, e.g., | 12 |
| | | 112.5 | |
| 60-89 | | 87.5-137.5, e.g., | 12 |
| | | 112.5 | |
| 30-59 | | 80-130, e.g., 105 | 12 or 24 |
| 10-29 | | 80-130, e.g., 105 | 12, 24 or 48 |
| ≥90 | 160 | 125-175, e.g., 150 | 12 |
| 60-89 | | 125-175, e.g., 150 | 12 |
| 30-59 | | 117.5-167.5, e.g., | 12 or 24 |
| | | 142.5 | |
| 10-29 | | 110-160, e.g., 135 | 12, 24 or 48. |

16. The method of claim 15, wherein:
a. the oral sotalol hydrochloride dosing regimen comprises 80 mg oral doses;
the creatinine clearance of the subject is in the range of 10-59 mL/min; and
the IV dose is administered in an amount of 67.5 mg; or
b. the oral sotalol hydrochloride dosing regimen comprises 120 mg oral doses;
the creatinine clearance of the subject is in the range of 10-59 mL/min; and
the IV dose is administered in an amount of 105 mg; or
c. the oral sotalol hydrochloride dosing regimen comprises 160 mg oral doses;
the creatinine clearance of the subject is in the range of 30-59 mL/min; and
the IV dosage is administered in an amount of 142.5 mg; or
d. the oral sotalol hydrochloride dosing regimen comprises 160 mg oral doses;
the creatinine clearance of the subject is in the range of 10-29 mL/min; and
the IV dose is administered in an amount of 135 mg.

17. The method of claim 16, wherein the subject/patient is currently in sinus rhythm, or is not currently in sinus rhythm, or has been or will be converted to sinus rhythm with sotalol or another antiarrhythmic and/or cardioversion, such as electric cardioversion (e.g., DCCV or direct current cardioversion), and sotalol hydrochloride is administered for the treatment of one or more cardiovascular condition selected from atrial flutter (AFL), atrial fibrillation (AF), persistent atrial fibrillation, paroxysmal atrial fibrillation, paroxysmal or persistent atrial fibrillation or atrial flutter, such as for patients who have experienced a recent AF/AFL episode who are in sinus rhythm or who will be cardioverted, ventricular fibrillation (VF) or ventricular arrhythmias, life-threatening recurrent VF or life-threatening recurrent hemodynamically unstable VT, focal atrial tachycardia, supraventricular tachycardia (SVT), atrioventricular re-entrant tachycardia (AVRT), atrioventricular nodal re-entrant tachycardia (AVNRT), atrial tachycardia, paroxysmal atrial tachycardia, junctional tachycardia, junctional ectopic tachycardia (JET), congenital heart disease (CHD), multifocal atrial tachycardia, atrial ectopic tachycardia, accessory pathway SVT, infantile SVT, post-operative SVT, paroxysmal SVT, treating Wolff-Parkinson-White syndrome, ventricular tachycardia (VT), premature ventricular contractions (PVCs), hemodynamically stable or unstable ventricular tachycardia, heart failure, coronary artery disease, and pulmonary artery hypertension, maintenance of normal sinus rhythm (for example, delay in time to recurrence of atrial fibrillation/atrial flutter (AF/AFL)) for example in patients with symptomatic AF/AFL and/or who are currently in sinus rhythm, and/or indicated for the treatment of life-threatening ventricular tachycardia; and/or treating people, such as hospitalized people, with atrial fibrillation cardioverted to normal sinus rhythm, without ventricular arrhythmias or various forms of blocks, in patients with normal kidney function.

18. The method of claim 17, wherein:
a. the oral sotalol hydrochloride dosing regimen comprises 80 mg oral doses;
the creatinine clearance of the subject is in the range of 10-59 mL/min; and
the IV dose is administered in an amount of 67.5 mg; or
b. the oral sotalol hydrochloride dosing regimen comprises 120 mg oral doses;
the creatinine clearance of the subject is in the range of 10-59 mL/min; and
the IV dose is administered in an amount of 105 mg; or
c. the oral sotalol hydrochloride dosing regimen comprises 160 mg oral doses;
the creatinine clearance of the subject is in the range of 30-59 mL/min; and
the IV dosage is administered in an amount of 142.5 mg; or
d. the oral sotalol hydrochloride dosing regimen comprises 160 mg oral doses;
the creatinine clearance of the subject is in the range of 10-29 mL/min; and
the IV dose is administered in an amount of 135 mg.

19. The method of claim 18, wherein the subject is currently in sinus rhythm and/or has experienced cardioversion to sinus rhythm, for example by way of electrocardioversion, such as DCCV, or chemical cardioversion, such as with sotalol hydrochloride, or combinations thereof.

20. The method of claim 19, wherein the selected interval is a 12-hour, 24-hour or 48-hour interval and the IV dose is a 5-hour intravenous dose.
